(19) United States
(12) Patent Application Publication
(10) Pub. No.: US 2025/0367475 A1
(43) Pub. Date: Dec. 4, 2025

(54) MULTI-ISOTOPE LOW-COUNT QUANTITATIVE SPECT METHOD FOR RADIOPHARMACEUTICAL

(71) Applicant: Washington University, St. Louis, MO (US)

(72) Inventors: Abhinav Kumar Jha, St. Louis, MO (US); Kweku Enninful, St. Louis, MO (US); Daniel Thorek, St. Louis, MO (US); Richard Wahl, St. Louis, MO (US); Zekun Li, St. Louis, MO (US)

(21) Appl. No.: 19/225,386

(22) Filed: Jun. 2, 2025

Related U.S. Application Data
(60) Provisional application No. 63/655,845, filed on Jun. 4, 2024.

(51) Int. Cl. A61N 5/10; G01T 1/164

(52) U.S. Cl. CPC A61N 5/1071; G01T 1/1642; A61N 2005/1021

(57) ABSTRACT

A computer system and method for providing single-photon emission tomography (SPECT) uptake data. A processor of the computer system is caused to receive SPECT data obtained from a SPECT acquisition, and quantify regional activity uptake of at least one isotope of a plurality of isotopes.

### CROSS-REFERENCE TO RELATED APPLICATION

This application claims priority to U.S. provisional application No. 63/655,845 filed on Jun. 4, 2024, the entire content and disclosures of which are incorporated herein by reference in their entireties.

### STATEMENT REGARDING FEDERALLY SPONSORED RESEARCH & DEVELOPMENT

This invention was made with government support under EB031962 awarded by the National Institutes of Health and under 2239707 awarded by the National Science Foundation. The government has certain rights in the invention.

### BACKGROUND

The field of disclosure relates to radiopharmaceutical therapies and more specifically the quantification of uptake.

Actinium-225 (Ac-225) is emerging as a promising candidate for targeted alpha therapy, necessitating the important need for methods to quantify absorbed dose in tumors and radio-sensitive organs. Ac-225 decay produces 7 emissions, providing a way to perform imaging-based dosimetry using single-photon emission computed tomography (SPECT). However, reliable Ac-225 quantification using conventional SPECT reconstruction-based quantification methods is challenging due to several reasons including extremely low number of detected counts, impact of stray-radiation noise, and image degrading effects in SPECT. Further, Ac-225 decays to multiple radioactive daughters, including Francium-221 (Fr-221) and Bismuth-213 (Bi-213), each of which can form independent biodistributions from Ac-225, and with crosstalk among the isotope emissions. Hence, there is a need for methods to jointly quantify the regional uptake of these isotopes.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF DESCRIPTION OF THE DISCLOSURE

One aspect of the present disclosure is a computer system for providing single-photon emission tomography (SPECT) uptake data. The computer system includes at least one processor in communication with at least one memory device. The at least one processor is programmed to receive SPECT data obtained from one or more SPECT scanners configured to perform at least one SPECT acquisition. The SPECT data includes uptake information associated with the at least one SPECT acquisition. The at least one processor is further programmed to quantify regional activity uptake of at least one isotope of a plurality of isotopes based on the uptake activity.

Another aspect of the present disclosure is a computer-implemented method for providing single-photon emission tomography (SPECT) uptake data using at least one processor in communication with at least one memory device. The method comprising: receiving SPECT data obtained from one or more SPECT scanners configured to perform at least one SPECT acquisition wherein the SPECT data includes uptake information associated with the at least one SPECT acquisition; and quantifying regional activity uptake of at least one isotope of a plurality of isotopes based on the uptake activity.

Yet another aspect of the present disclosure is one or more non-transitory computer-readable storage media for a computing system providing single-photon emission tomography (SPECT) uptake data. The one or more non-transitory computer-readable storage media comprises a plurality of instructions stored thereon that, in response to being executed, cause the computing system to: receive SPECT data obtained from one or more SPECT scanners configured to perform at least one SPECT acquisition wherein the SPECT data includes uptake information associated with the at least one SPECT acquisition; and quantify regional activity uptake of at least one isotope of a plurality of isotopes based on the uptake activity.

### BRIEF DESCRIPTION OF THE DRAWINGS

Various refinements exist of the features noted in relation to the above-mentioned aspects. Further features may also be incorporated in the above-mentioned aspects as well. These and other features, aspects, and advantages of the present disclosure will become better understood when the following detailed description is read with reference to the accompanying drawings in which like characters represent like parts throughout the drawings.

FIG. 1. is an energy spectra of Ac-225, Fr-221 and Bi-213.

FIG. 2. is an activity map of a modelled patient.

FIG. 3. demonstrates a comparison of the simulated and physical scanned SPECT projections of NEMA phantom in Ac-225, Fr-221 and Bi-213 energy windows.

FIG. 4. illustrates the absolute normalized bias (NB), normalized standard deviation (NSD), and normalized root mean squared error (NRMSE) of the estimated lesion uptake of Ac-225, Fr-221 and Bi-213 as a function of lesion diameter.

FIG. 5 illustrates the absolute NB, NSD, and NRMSE of the estimated lesion uptake of Ac-225, Fr-221 and Bi-213 as a function of lesion contrast.

FIG. 6 illustrates the absolute NB, NSD, and NRMSE of the estimated lesion uptake of Ac-225, Fr-221 and Bi-213 as a function of % retention of daughter isotopes.

FIG. 7 illustrates the NSD obtained using a method disclosed herein compared with that derived from the Cramer-Rao Lower bound.

FIG. 8 illustrates the absolute NB and NRMSE of the estimated lesion uptake of Ac-225, Fr-221 and Bi-213 for all considered cases of inaccurate volume of interest (VOI) definitions.

FIG. 9 illustrates the absolute NB and NRMSE of the estimated lesion uptake of Ac-225, Fr-221 and Bi-213 for various levels of heterogeneity in the activity distribution.

FIG. 10 illustrates a process diagram for providing single-photon emission computed tomography (SPECT) uptake data.

FIG. 11 illustrates a schematic diagram of a SPECT system.

FIG. 12. is a block diagram of an example computing device.

Unless otherwise indicated, the drawings provided herein are meant to illustrate features of embodiments of the disclosure. These features are believed to be applicable in a wide variety of systems including one or more embodiments of the disclosure. As such, the drawings are not meant to include all conventional features known by those of ordinary skill in the art to be required for the practice of the embodiments disclosed herein.

### DETAILED DESCRIPTION

The following detailed description illustrates embodiments of the present disclosure to enable one skilled in the art to make and use the disclosure, describes several embodiments, adaptations, variations, alternatives, and uses of the disclosure. The disclosure includes systems and methods to improve radiotherapy of a subject. As used herein, a subject is a human, an animal, or a phantom, or part of the human, the animal, or the phantom such as an organ or part of an organ.

Alpha-particle radiopharmaceutical therapies (α-RPTs) have gained popularity in the past few years. Since α particles have a short range in tissues and high linear energy transfer, α-RPTs can cause significant damage to cancer cells and minimal damage to healthy tissues. Radium-223 was approved as the first α-RPT and since there has been an increase in interest in alpha emitters for several cancers including neuroendocrine tumors, prostate, breast, colon, and ovarian cancer. Despite the promise of α-RPTs, progress is needed in many aspects of its development. For instance, the administration of radiopharmaceuticals results in a distribution of the radionuclide throughout the subject, and this is true for α-RPTs as well. Also, some α-RPTs produce daughter isotopes that emit a and/or p particles.

Even though chelators are used in targeting the isotopes, it is often observed with decays chains that have multiple alpha emissions that the first daughter will get displaced from the chelate due to the recoil from the first alpha emission. The daughter isotopes that follow could then diffuse from the targeting molecule, potentially resulting in independent biodistributions of the isotopes in the subject which can cause radiotoxicity in organs at risk. As such, reliable dosimetry is important to monitor the levels of accumulation at disease sites and critical organs and predict treatment outcomes. Most α-emitting isotopes also emit X and γ rays which can be detected using a γ camera. This provides a way to use quantitative single-photon emission computed tomography (SPECT) imaging methods to quantify activity uptake in regions of interest. The estimated activity uptake can then serve as input for dosimetry. However, reliable quantification using conventional quantitative SPECT methods for many α-RPTs has been challenging. This is due to the low number of detected counts, the presence of multiple photopeak due to the formation of daughter isotopes, influence of background noise and image degrading artifacts in SPECT.

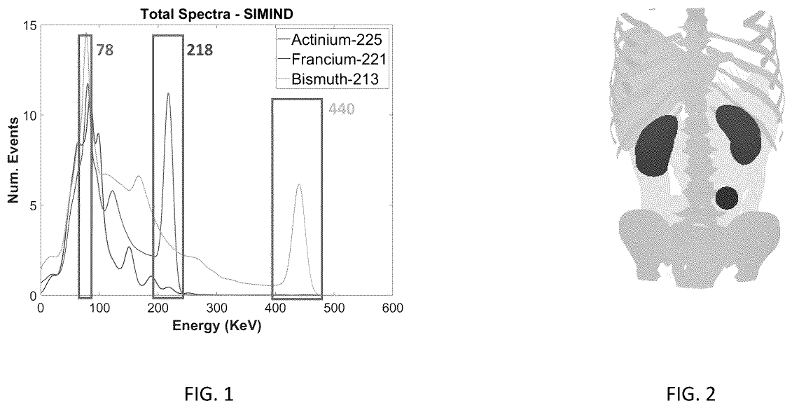

Ac-225 is a pure α-emitter with a half-life of 9.9 days. A single Ac-225 decay yields four α, two β-particles and γ emissions which can be detected by a γ camera. Ac-225 decay produces seven daughter radionuclides including Francium-221 (Fr-221) and Bismuth-213 (Bi-213) and decays to a stable Bismuth-209. Fr-221 (half-life 4.9 min) decay yields an α-particle whilst Bi-213 (half-life 46 min) also yields alpha and beta particles in its decay. As seen in FIG. 1, the three isotopes, Ac-225, Fr-221 and Bi-213 produce γ-emissions at 78, 218 and 440 KeV energy windows, respectively. This provides a way to monitor subject response to treatment and the distribution of Ac-225 and its daughters (Fr-221 and Bi-213) throughout the subject using SPECT imaging and reliable quantification.

Conventional quantitative SPECT methods use reconstruction-based approaches. However, for estimating multiple isotopes, compensation using noisy crosstalk estimates amplifies image noise and reconstruction-based methods have generally been shown to have very limited accuracy and precision at low counts. In reconstruction-based approaches, activity uptake in regions of interest is estimated by averaging over the activity of all the voxels in a volume of interest(s). This means, estimations are done for many voxels using the projection data. Given that this is an inherently ill-posed problem, it becomes much more challenging with low projection counts.

To circumvent these challenges, Projection Domain Quantification (PDQ) methods have been proposed to make estimates of regional activity uptake directly from projection data. Also proposed has been low-count quantitative SPECT (LC-QSPECT) and the multiple-energy-window projection-domain quantitative (MEW-PDQ) SPECT methods. The LC-QSPECT method uses projection data from a single photopeak window to estimate the regional activity uptake of a radioisotope. The MEW-PDQ method advances on the LC-QSPECT method to use projection data from multiple energy windows and estimate the regional activity uptake of two isotopes. These PDQ methods avoid the ill-posed nature of reconstruction by estimating activity uptake in specified volume of interests (VOIs) which would usually correspond to the regions of interest. Even though LC-QSPECT and MEW-PDQ methods have been shown to perform significantly better than reconstruction-based methods, a major setback of these methods is that they are not applicable in cases where there are more than two γ-emitting isotopes present in the subject that need to be monitored as it is for isotopes like Actinium-225 (Ac-225).

Therefore, there is a need for a generalized framework to perform reliable activity uptake estimations of multiple isotopes using SPECT projection data. In this context, the approach of this disclosure utilizes a generalized Multi-Isotope Low-Count Quantitative SPECT (MI-LC-QSPECT) method. The disclosed MI-LC-QSPECT method directly estimates the regional activity uptake of any number of γ-emitting isotopes using the SPECT projections acquired over multiple energy windows. For a certain number of isotopes, derived is a series of equations, one for each isotope, that model the crosstalk among the isotope emissions and are solved iteratively to estimate the regional activity uptake of each isotope. This method was then validated by performing activity uptake quantification for Ac-225.

### Methodology

Systems and methods disclosed herein directly estimate the regional activity uptake of any number of γ-emitting isotopes using the single-photon emission computed tomography (SPECT) projections acquired over multiple energy windows. Specifically, to address the previously disclosed challenges, disclosed is a generalized Multi-Isotope Low-Count Quantitative SPECT (MI-LC-QSPECT) method. MI-LC-QSPECT directly estimates regional activity uptake of multiple γ-emitting isotopes using SPECT projections from various energy windows. For a certain N number of isotopes, a series of N-equations that model the crosstalk among the isotope emissions was derived. The equations are solved iteratively to estimate the regional activity uptake of each isotope using the measured projection data and the system response matrix of the respective isotopes. To evaluate this method, realistic simulation studies were conducted in the context of subjects with neuroendocrine tumors treated with 12 MBq of actinium-225 (Ac-225)-based peptide receptor radionuclide therapy (PRRT). 3D digital subjects generated with the extended cardiac-torso (XCAT) phantom were imaged with a Siemens Symbia™ SPECT system with high-energy general-purpose (HEGP) collimator, simulated using the SIMIND Monte Carlo simulation software, following clinically relevant protocols.

All relevant image-degrading processes were modeled. Projections were acquired in 20% primary energy windows centered on 78, 218 and 440 keV, corresponding to photopeaks of Ac-225, Francium-221 (Fr-221) and Bismuth-213 (Bi-213) respectively (see FIG. 1). Projections were acquired at 64 evenly distributed angular positions in 32 minutes. The generalized multi-isotope low-count quantitative single-photon emission computed tomography (MI-LC-QSPECT) method was used to estimate the mean regional uptake of all three isotopes for organs with significant uptake, the lesion, and the rest of the body (background). The accuracy and precision of the method was evaluated for different lesion sizes, lesion to gut contrasts and uptake retention rates of the daughter isotopes in the lesion and kidney. The method was compared with a projection-domain low count quantitative single-photon emission computed tomography method (LC-QSPECT) that does not model crosstalk and a dual-isotope ordered subset expectation maximization (DOSEM)-based reconstruction method for Fr-221 and Bi-213.

The disclosed method represents an advancement in quantitative SPECT imaging of α-RPTs. An important feature of MI-LC-QSPECT is the modelling of crosstalk among multiple isotopes. Crosstalk, arising from the overlap of energy spectra, spatial distributions or scattered photons, can significantly affect the accuracy of uptake estimations. By explicitly accounting for crosstalk, MI-LC-QSPECT offers precise and reliable estimations, even in scenarios where multiple isotopes coexist. The method also has similar advantages as proposed PDQ methods such as LC-QSPECT and MEW-PDQ. By estimating a much lower number of parameters, which is the regional uptake in K VOIs, MI-LC-QSPECT avoids the issue of ill-posed-ness associated with reconstruction-based quantification methods, irrespective of the number of isotopes involved. The MI-LC-QSPECT method also directly estimates regional uptake from projection data using defined boundaries of VOIs which are obtained from high resolution images. This significantly minimizes the errors associated with partial volume effects (PVEs) and noise induced bias associated with reconstruction. As disclosed herein, this approach reduces computational complexity and enhances the accuracy of activity uptake estimations, particularly in low-count settings.

Consider a SPECT system imaging a radioisotope distribution of multiple isotopes. The radioisotopes produce γ emissions at multiple energies. The photon source distribution is denoted as Ξ(r,ε), where r=(x,y,z) denote the spatial 3D coordinates and ε represents the energy of emitted γ ray photons. Denote the measured projection data by an M-dimensional vector, g, where M=Total number of projection bins for all spatial and angular locations×number of energy windows.

Given that the object being imaged, and the projection data lie in the Hilbert space of square integrable functions, denoted by [symbol] ₂( [symbol] ⁴), and the Hilbert space of Euclidean vectors, denoted by [symbol] ^{M}, the imaging of the photon source distribution by the SPECT system, denoted by the operator [symbol] can be described as an integral transform from [symbol] ₂( [symbol] ⁴) to [symbol] ^{M}. Denote the kernel of the [symbol] operator as hₘ(r, ε), which defines the system response of the mth element of the projection g to a photon emitted from position r with energy ε.

In the context of SPECT imaging for α-RPT, stray radiation-related noise contributes significantly to the measured counts due to the low number of photon count from the subject. Stray radiation-related noise refers to noise acquired from detected photons which are emitted from areas other than the subject. Modeled is stray radiation-related noise as a Poisson distribution denoted by the M-dimensional vector, Ψ with each element ψₘ, denoting the mean of the stray radiation-related noise at each energy window. Let n be an M-dimensional vector representing the noise in the imaging system. Therefore, the imaging system equation is given by:

$$g = Ξ + Ψ + n \qquad ( 1 )$$

The term Ξ(r,ε) consists of γ-emissions from all isotopes being imaged. Let J denote the number of isotopes, so the term can be defined as:

$$Ξ ( r , ε ) = {\sum }_{j = 1}^{J} {Ξ}^{j} ( r , ε ) , \qquad ( 2 )$$

where Ξ^{j}(r,ε) represents γ-emissions from the j^{th} isotope. Since emission rates are independent of spatial location, Equation 2 can be further decomposed to:

$$Ξ ( r , ε ) = {\sum }_{j = 1}^{J} {f}^{j} ( r ) {ζ}^{j} ( ε ) , \qquad ( 3 )$$

where ζ^{j}(ε) describes the mean emission rates of γ-ray photons with energy ε from a unit activity of the j^{th} isotope and ƒ^{j}(r) represent the spatial distribution of the j^{th} isotope. The imaging system equation can now be written as:

$${g}_{m} = {\sum }_{j = 1}^{J} \int {f}^{j} ( r ) \int {h}_{m} ( r , ε ) {ζ}^{j} ( ε ) d ε {d}^{3} r + {ψ}_{m} + {n}_{m} \qquad ( 4 )$$

$$Let {h}_{m}^{j} ( r ) = \int {h}_{m} ( r , ε ) {ζ}^{j} ( ε ) d ε , so that ,$$

$${g}_{m} = {\sum }_{j = 1}^{J} \int {h}_{m}^{j} ( r ) {f}^{j} ( r ) {d}^{3} r + {ψ}_{m} + {n}_{m} \qquad ( 5 )$$

Since the interest is in estimating the regional uptake within a set of VOIs, the 3-D VOI mask will be denoted by the function

$${ϕ}_{k}^{VOI}$$

(r), where:

$${ϕ}_{k}^{VOI} ( r ) = \{ \begin{matrix}1 , & if r lies within the {k}^{t} VOI \\ 0 , & otherwise\end{matrix} \qquad ( 6 )$$

Denote λ^{j} as a K-dimensional vector of regional uptake for the j^{th} isotope. ƒ^{j}(r) is now represented in terms of the VOI basis functions as:

$${f}_{VOI}^{j} ( r ) = {\sum }_{j = 1}^{J} {\sum }_{k = 1}^{K} {λ}_{k}^{j} {ϕ}_{k}^{VOI} ( r ) , \qquad ( 7 )$$

where

$${f}^{j} ( r ) = {f}_{VOI}^{j} ( r )$$

if the activity in each VOI is constant. With this representation for ƒ^{j}(r), the expression for the m^{th} element of the vector g is given by:

$${g}_{m} = {\sum }_{j = 1}^{J} {\sum }_{k = 1}^{K} {λ}_{k}^{j} \int {h}_{m}^{j} ( r ) {ϕ}_{k}^{VOI} ( r ) {d}^{3} r + {ψ}_{m} + {n}_{m} \qquad ( 8 )$$

This can also be written in a more compact form as:

$$g = {\sum }_{j = 1}^{J} {H}^{j} {λ}^{j} + Ψ + n \qquad ( 9 )$$

Where H^{j} is an M×K-dimensional system matrix with elements given by:

$${H}_{mk}^{j} = \int {d}^{3} {rh}_{m}^{j} ( r ) {ϕ}_{k}^{VOI} ( r ) \qquad ( 10 )$$

Which can be even further simplified by defining H=[H¹ H² . . . H^{j}] and

$$λ = [ \begin{matrix}{λ}^{1} \\ {λ}^{2} \\ ⋮ \\ {λ}^{J}\end{matrix} ]$$

to give:

$$g = H λ + Ψ + n \qquad ( 11 )$$

To estimate λ given g, the probability of occurrence of the measured data was maximized. Let Pr(x) be the probability of a Poisson distributed discrete random variable x. Then the likelihood of the measured projection data is given as:

$$Pr ( g \vert λ ) = {\prod }_{m = 1}^{M} Pr ( {g}_{m} \vert λ ) \qquad ( 12 )$$

To estimate λ, the log likelihood of λ given g was maximized. In other words, the log likelihood with respect to λ was differentiated and the point that maximizes the log likelihood was found by equating the differential to zero. This process can be expressed simply as:

$$\hat{λ} = arg {max}_{λ} ln [ Pr ( g \vert λ ) ] \qquad ( 13 )$$

This can be solved iteratively using the maximum-likelihood expectation maximization (MLEM) algorithm, yielding a series of J equations:

$${\hat{{λ}^{1}}}_{k}^{( t + 1 )} = {\hat{{λ}^{1}}}_{k}^{( t )} \frac{1}{{\sum }_{m = 0}^{M} {H}_{mk}^{1}} {\sum }_{m = 1}^{M} \frac{{g}_{m}}{{( H {\hat{λ}}^{( t )} )}_{m} + {ψ}_{m}} {H}_{mk}^{1} \qquad ( 14 - 1 )$$

$${\hat{{λ}^{2}}}_{k}^{( t + 1 )} = {\hat{{λ}^{2}}}_{k}^{( t )} \frac{1}{{\sum }_{m = 0}^{M} {H}_{mk}^{2}} {\sum }_{m = 1}^{M} \frac{{g}_{m}}{{( H {\hat{λ}}^{( t )} )}_{m} + {ψ}_{m}} {H}_{mk}^{2} \qquad ( 14 - 2 )$$

$${\hat{{λ}^{J}}}_{k}^{( t + 1 )} = {\hat{{λ}^{J}}}_{k}^{( t )} \frac{1}{{\sum }_{m = 0}^{M} {H}_{mk}^{J}} {\sum }_{m = 1}^{M} \frac{{g}_{m}}{{( H {\hat{λ}}^{( t )} )}_{m} + {ψ}_{m}} {H}_{mk}^{J} \qquad ( 14 - J )$$

To implement the method, it is first needed to obtain

$${H}_{m}^{j} ,$$

which denotes the system response matrices of each of the isotopes being quantified. This can be done by using a Monte Carlo (MC)-based simulation software like SIMIND. The emission spectra of the γ and X-ray photons of each of the isotopes are modeled in this approach. The system response matrices from all the photopeak energy windows are obtained for each isotope which will now become the elements of H. Thus, now exists the system response matrix from all the photopeak energy windows of each isotope enabling us to account for crosstalk contamination present in each energy window. This allows estimation of activity uptake within specific VOIs, which can be defined using segmentations from X-ray CT scans or a combination of X-ray CT and SPECT scans. With this, the system only needs to generate the system response matrices pertaining to the VOI definitions denoted by

$${H}_{m k}^{j}$$

in Equation 10. As disclosed earlier, stray radiation-related noise is accounted for in this method. To obtain the stray radiation-related vector Ψ, planar blank scans can be acquired using a similar imaging system and protocol as will be used for the subject.

### Examples

To evaluate MI-LC-QSPECT, it was necessary to implement it in a setup where the ground truth of activity uptake in ROIs is known. To achieve this, a study was conducted to assess a realistic simulation in the context of subjects with primary tumor in the gut and a total activity uptake of 12 MBq of Ac-225-based peptide receptor radionuclide therapy (PRRT) in the torso. The subject had significant uptake in the bladder, spleen, gut, liver, bone marrow and kidney. The rest of the organs had similar low uptakes and so are grouped together as background. The radioisotope uptake in each VOI was distributed according to the ratio as shown in Table 1 below. Imaging for the subjects also occurred after secular equilibrium which means the Ac-225, Fr-221 and Bi-213 had similar activities. For simplicity a similar radioisotope spatial distribution for Ac-225, Fr-221 and Bi-213 was simulated.

| | VOI 2 | | | VOI 5 | | |
|---|---|---|---|---|---|---|
| VOI 1 | Spleen & | VOI 3 | VOI 4 | Bone | VOI 6 | VOI 7 |
| Background | Bladder | Gut | Liver | marrow | Kidneys | Lesion |
| 1 | 5 | 12 | 22 | 25 | 74 | 360 |

FIG. 2 illustrates an example anthropomorphic digital phantoms of a standard male subject torso were generated using the Extended Cardiac-Torso (XCAT) phantom (refer to W. P. Segars, M. Mahesh, T. J. Beck, E. C. Frey, and B. M. W. Tsui, “Realistic CT simulation using the 4D XCAT phantom,” Med Phys, vol. 35, no. 8, 2008, doi: 10.1118/1.2955743). Then, activity and attenuation maps with an axial dimension of 512×512 and 364 slices defined the depth were generated. The side length per voxel was 1.105 mm. This simulates continuously distributed radioisotope activity and attenuation coefficient.

Next, the imaging process using the SIMIND Monte-Carlo program was simulated. The simulation was done in the context where the subject's torso was imaged using a Siemens™ Symbia SPECT/CT Imaging system equipped with NaI(TI) crystals and parallel high-energy general-purpose (HEGP) collimator. The inherent spatial and energy resolution of the scintillation detector in the system measured 3.9 mm and 9.8% at 140 keV, respectively. Sixty-four projections taken at 30 secs per angular projection were acquired in 20% primary energy windows centered on 78, 218 and 440 KeV, which corresponds to photopeak of Ac-225, Fr-221 and Bi-213 respectively (as shown in FIG. 1). The angular positions of the projections were uniformly spaced over 360-degrees. The stray radiation related noise was modelled based on experimental values obtained previously (refer to Z. Li et al., “A Projection-Domain Low-Count Quantitative SPECT Method for α-Particle-Emitting Radiopharmaceutical Therapy,” IEEE Trans Radiat Plasma Med Sci, vol. 7, no. 1, 2023, doi: 10.1109/TRPMS.2022.3175435). All relevant image-degrading processes, including attenuation, scatter, and collimator-detector response, were also modeled. The emission spectra of all three isotopes were simulated. Multiple imaging instances (noise realizations) were generated for each simulated phantom.

Afterwards, the accuracy of the simulations was validated by comparing projection data obtained using SIMIND simulations with that obtained on an actual Siemens Symbia™ SPECT/CT scanner. To do this, projections, acquired from a NEMA phantom with six spheres. Each sphere was filled with 35 KBq/ml of Ac-225 and the main cavity of the phantom body was filled with water. Body-contour acquisition orbit was utilized, 60 projections were acquired with each angular position uniformly spaced over 360-degrees. The acquisition time for each projection was 30 s and the HEGP collimator was used. For this acquisition, the energy windows were centered on 82 keV, 216.8 keV and 444.3 keV with window width of 20%, 8% and 5% respectively. The acquisition was modeled using the simulation approach. The activity map simulated the known Ac-225 activity used for the phantom, and the activities of Bi-213 and Fr-221 were similar to that of Ac-225 due to secular equilibrium. The attenuation map was obtained from the CT scans. The projection data in the three energy windows from the simulation and those obtained with the physical scanner were compared.

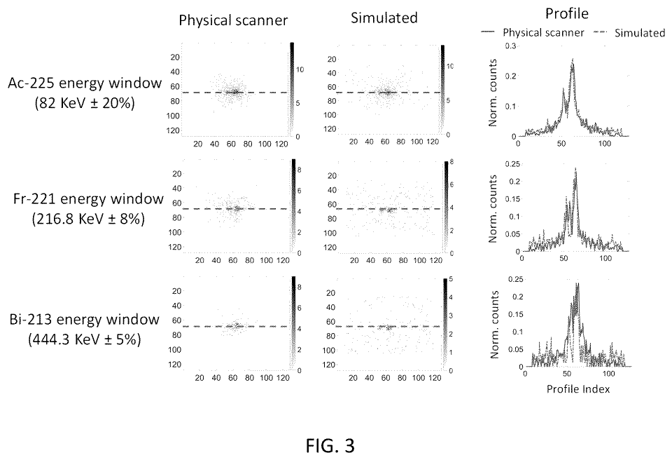

FIG. 3 illustrates projections of the NEMA phantom in the Ac-225, Fr-221 and Bi-213 energy windows at the first angular position acquired from the simulations and the physical scanner. The profiles of the projections along the dashed line were also compared. To minimize noise-related variation in the profiles, first the projection counts were normalized, and each point was generated in the profile by averaging over ten adjacent pixels on both sides of the dashed line. It can be observed that the profile of the simulated projection matched that of the physical scanner in all the energy windows, providing evidence of the accuracy of the simulation approach.

To prove the efficacy of the disclosed method, the performance of MI-LC-QSPECT in different scenarios was evaluated. The accuracy and precision of the disclosed method with various lesion sizes, lesion contrasts and uptake retention rates of the daughter isotopes Fr-221 and Bi-213 in the lesion was also evaluated. For these experiments, the conditions where the VOIs had homogenous uptake and no inaccuracy in the VOI definitions were maintained. Then, the disclosed method in the conditions where there were inaccurate VOI definitions and intra-regional heterogenous activity-uptake distributions. Projections were obtained for the subject phantoms as earlier described.

### Different Lesion Sizes and Contrasts

To evaluate performance of MI-LC-QSPCET for various lesion sizes, five subject phantoms with lesion diameters of 15 mm, 20 mm, 25 mm, 30 mm, and 35 mm respectively were generated. For lesion contrasts, the ratio of uptake in the lesion and gut were variated. Five subject phantoms were also generated for this experiment, however this time each subject had the same lesion diameter of 33.75 mm and lesion to gut ratios of 2:1, 3:1, 4:1, 5:1 and 6:1 respectively. These ratios are referred to as lesion contrasts from here onwards. As shown in Table 1, the uptake ratios of the other VOIs however remained. 100 noise realizations were generated for each subject.

### Rate of Retention

In α-RPTs, isotopes are bound to chelators to target them to the site of the cancerous cells. Often, daughters of decays chains with multiple alpha emissions get displaced from the chelate, diffusing into the blood and creating independent biodistributions apart from the parent isotope. Regions like the lesion, with high concentrations of the parent isotope, may not retain as much free daughters. The performance of the proposed method for various retention rates of the daughter isotopes is evaluated. This is to investigate usage of the disclosed method in accurately monitoring different levels of activity uptake of multiple isotopes in the same region. This also informs the presence of an isotope in an organ at risk and helps to identify daughter isotopes with distinct biodistributions from the parent. The retention rates of Fr-221 and Bi-213 from 0 to 100% were considered with 20% increments. The retention rate of Ac-225 remained at 100% for each simulation. Subject phantoms were generated for each retention rate of the daughters considered. 100 noise realizations were generated for each subject.

### VOI Definition Inaccuracies

In the clinical setting, VOIs are normally obtained from the CT scans of subjects. Inaccuracies in the VOI definitions may be observed when aligned with SPECT data. Factors which can contribute to such inaccuracies are subject movement during SPECT scans and movement of organs caused by respiratory motions in the subject. The performance of MI-LC-QSPECT with these inaccuracies present were evaluated. First considered were inaccuracies caused by subject movement during SPECT scans. To simulate this, a standard subject with a lesion size of 33.75 mm whose VOIs have been defined from an initial CT scan was modeled. Afterwards, subject movement before taking the SPECT scan was simulated. Three movement scenarios were modeled, (i) shifting of the subject sideways (along the x-axis) in both left and right directions, (ii) shifting of the subject vertically (along the y-axis) in both forward and backwards directions and (iii) rotation of the subject in both directions around the y-axis. The subject was shifted by 2, 4 and 6 voxels and rotated by 1 and 2 degrees. The levels of misalignment were chosen based on reported values of misregistration of CT and SPECT scans observed after performing registration. 50 noise realizations of the subject were generated for each simulated level of misalignment.

Then the proposed method with inaccuracies generated through the motions of the organs during respiration was evaluated. To simulate this, it was first assumed that there has been little to no movement of the subject's organs during the CT scan for VOI definition. Next, the same standard subject as was done when simulating subject movement was modeled. For this scenario however, a subject breathing with 5 secs respiratory cycle during the SPECT scan was used. This was done using X-CAT. Five activity and attenuation maps, one for each second in the respiratory cycle were generated. These represented frames of movement during respiration. Projections were obtained of each frame following the imaging procedure described earlier and generated the projection for one respiratory cycle using the average of the projections from the frames. 50 noise realizations of the projection for one respiratory cycle were generated and estimations were performed using those projections and initially acquired VOI definitions.

### Intra-Regional Heterogenous Activity Uptake Distribution

As discussed previously, the MI-LC-QSPECT method assumes a homogenous activity uptake distribution of the isotopes in the VOIs. This may however not be the case in clinical scenarios. Often, the isotope distribution within organs of interest in the subject are heterogenous. Using both CT and reconstructed SPECT images can help identify regions with relatively homogenous uptake, but due to SPECT's limited resolution and high noise levels, minor intra-regional uptake differences may still be hidden, and some level of heterogeneity could persist within defined areas. As such, assessing MI-LC-QSPECT's performance given heterogenous uptake distributions is necessary. To assess this performance, intra-regional heterogeneity was modeled in all the VOIs defined in Table 1. This was done by using a 3D lumpy model as defined as:

$${f}_{k} ( r ) = {ϕ}_{k}^{VOI} ( r ) {\sum }_{n = 1}^{N} \frac{{a}_{k}}{{( {σ}_{k} \sqrt{2 π} )}^{3}} exp ( - \frac{{( r - {c}_{n} )}^{2}}{2 {σ}_{k}^{2}} ) , \qquad ( 15 )$$

Where ƒₖ(r),

$${ϕ}_{k}^{VOI}$$

(r), αₖ and σₖ denote the activity distribution, support of the VOI, magnitude, and lump width for a defined region, k, respectively. N denotes the total number of lumps and cₙdenotes the center of the n^{th} lump, which is sampled from a uniform distribution.

The performance of the proposed method was determined across various levels of heterogeneity. To determine the level of heterogeneity, the mixture model was used, where the activity distribution in a VOI denoted as

$${f}_{𝒲}^{Mix}$$

(r) is defined by the sum of a scaled heterogenous distribution and a scaled homogenous distribution of the VOL. This can be written as:

$${f}_{𝒲}^{Mix} ( r ) = 𝒲 {f}^{het} ( r ) + ( 1 - 𝒲 ) {f}^{hom} ( r ) , \qquad ( 16 )$$

Where ƒ^{het}(r) denotes a heterogenous radioisotope distribution within a specified VOI, ƒ^{hom}(r) denotes a homogenous radioisotope distribution within that VOI and W denotes the scale of heterogeneity in the mixture model VOL. The values of W chosen were 0.2, 0.4, 0.6, 0.8 and 1. Five subjects phantoms at each considered intra-regional heterogeneity were generated. Each of these subjects had the same lesion diameter of 33.75 mm, standard total activity uptake in the torso and uptake ratios as defined in Table 1. Additionally, 50 noise realizations were generated for each of the simulated subjects.

To provide a more comprehensive understanding of the performance of the present invention, the performance of MI-LC-QSPECT was compared to (i) a dual-isotope ordered subset expectation maximization (DOSEM)-based reconstruction method and, (ii) a projection-domain low count quantitative SPECT method (LC-QSPECT) in all the experiments. This was done to not only validate the efficacy of MI-LC-QSPECT, but to identify areas where the proposed method offers advancements over these current methods.

### LC-QSPECT

To better understand the impact of modelling crosstalk contamination for multi-isotope estimations, the performance of the disclosed method with LC-QSPECT was compared. LC-QSPECT estimates the regional activity of a single isotope using the projection data from its photopeak window without modelling crosstalk contamination from other isotopes. Estimations of Ac-225, Fr-221 and Bi-213 were generated using the VOI definitions and projection data generated for each noise realization of the subject in all the experiments performed.

### Dual-Isotope Ordered Subset Expectation Maximization (DOSEM)-Based Reconstruction

There have been some recent advancements in quantitative imaging of Ac-225 where reconstruction-based methods have been used towards quantification of Ac-225 and its daughters. For example, a dosimetry sub study conducted as part of the ACTION-1 trial focusing on the imaging and dosimetry of Ac-225 employed a dual-radionuclide quantitative SPECT reconstruction technique to obtain activity images of Fr-221, acting as a surrogate for Ac-225, and Bi-213 using SPECT/CT images acquired at various time points post-infusion. This approach allows for the simultaneous quantification of Fr-221 and Bi-213. Specifically, one such dual-radionuclide quantitative SPECT reconstruction technique is the DOSEM-based reconstruction method (refer to H. de Jong, F. Beekman, M. Viergever, and P. van Rijk, “Simultaneous 99mTc/201T1 dual-isotope SPET with Monte Carlo-based down-scatter correction,” Eur J Nucl Med Mol Imaging, vol. 29, no. 8, pp. 1063-1071, Aug. 2002, doi: 10.1007/s00259-002-0834-1). This method was implemented by obtaining projections from the Fr-221 and Bi-213 energy windows which are centered on 218 and 440 KeV with 20% window width. The images were reconstructed using the ordered subset expectation maximization (OSEM) method which was implemented using the Customizable and Advanced Software for Tomographic Reconstruction (CASToR) software.

Crosstalk contamination was accounted for in this process. To estimate how much crosstalk contamination was in each energy window reconstructed images were first generated of each isotope using the projections obtained from their respective energy windows. The reconstructions are obtained with all compensations performed but crosstalk contamination compensation. The images of each isotope are then forward projected to the energy window of the other isotope to estimate the crosstalk contamination in that energy window. The emission spectra, attenuation, scatter, and collimator-detector response are modeled in performing the forward projection. Scatter compensations and crosstalk contamination from scatter photons was modelled using the effective scatter source estimation (ESSE) method, where SIMIND simulations were used in generating the scatter kernels. Having obtained the estimations of the crosstalk contamination in each energy window, the images of both isotopes could now be reconstructed.

As can be observed in FIG. 1, there are very minimal photons from Fr-221 and Ac-225 which end up in the 440 KeV window, therefore compensation for crosstalk contamination for Bi-213 reconstruction was not considered. This also gave the added benefit of avoiding excessive amplification of image noise which could originate from extremely noisy crosstalk estimates in the Bi-213 energy window. OSEM reconstructions were performed and compensated for attenuation, scatter, collimator detector response and stray-radiation-related noise with the estimated crosstalk contamination from Bi-213 in Fr-221's energy window as an additional corrective term for reconstruction of Fr-221. The OSEM reconstructions were performed using 3 iterations 16 subsets which was determined as optimal for activity recovery and minimal noise in the reconstructions. This was done for each noise realization of the projections obtained for Fr-221 and Bi-213 in all the experiments performed.

The goal of the study is to evaluate the accuracy, precision, and overall error of MI-LC-QPSECT as compared to DOSEM-based reconstruction and LC-QPSECT, therefore calculated was the normalized bias (NB), normalized standard deviation (NSD) and normalized root mean squared error (NRMSE) of estimations performed over multiple noise realizations of the modeled subject. Given N-number of noise realizations, the NB of the k^{th} VOI can be defined as:

$${NB}_{k} = \frac{1}{N} {\sum }_{n - 1}^{N} \frac{{\hat{λ}}_{nk} - {λ}_{nk}}{{λ}_{k}} , \qquad ( 17 )$$

Thus, the NSD of the k^{th} VOI as is also defined as:

$${NSD}_{k} = \sqrt{\frac{1}{N - 1} {\sum }_{n - 1}^{N} {( \frac{{\hat{λ}}_{nk}}{{λ}_{k}} - \frac{1}{N} {\sum }_{{n}^{′} = 1}^{N} \frac{{\hat{λ}}_{{n}^{′} k}}{{λ}_{k}} )}^{2}} , \qquad ( 18 )$$

where λₖ denotes the true activity uptake in the k^{th} VOI and \hat{λ}ₙₖ denotes its corresponding estimate for the n^{th} noise realization. The NRMSE of the k^{th} VOI was calculated using the root of the sum of the squared NB and NSD. This can be defined as:

$${NRMSE}_{k} = \sqrt{{NB}_{k}^{2} + {NSD}_{k}^{2}} \qquad ( 19 )$$

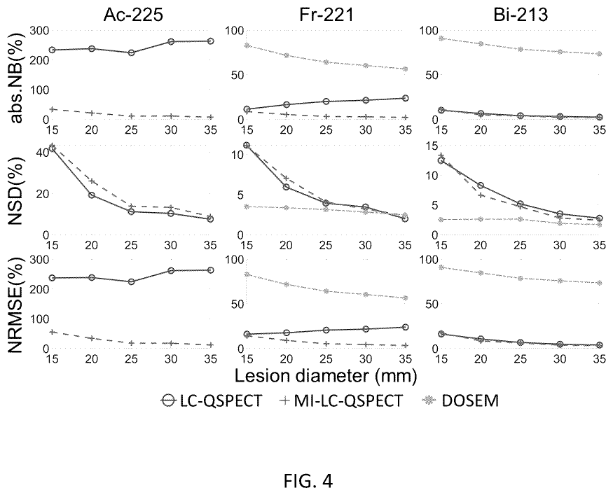

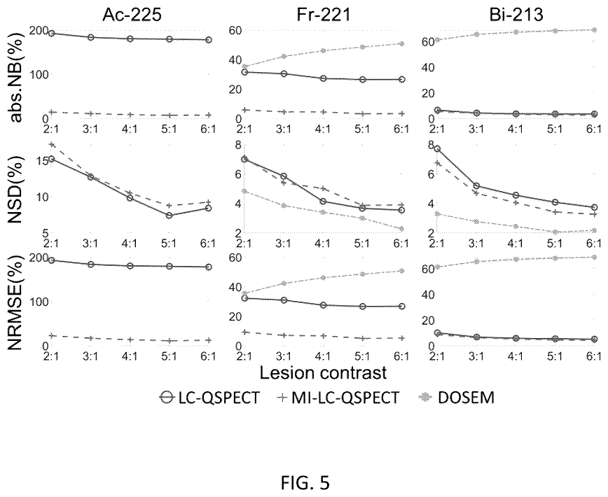

FIGS. 4-6 illustrate the absolute NB, NSD and NRMSE of the (i) estimated lesion uptake for the various lesion sizes, (ii) lesion contrasts, and (iii) retention rates of daughter isotopes. MI-LC-QSPECT achieves the best performance for all three isotopes, significantly outperforming both LC-QSPECT and DOSEM-based reconstruction. The disclosed method is observed to have relatively low sensitivity to PVEs as compared to DOSEM-based reconstruction. This is demonstrated in the accuracy of MI-LC-QSPECT for small lesion sizes. The challenges of using reconstruction-based methods, including PVEs, blurring and noise induced bias associated with reconstruction-based quantification methods in low count settings is made more apparent when the trend in performance of DOSEM-based reconstruction is observed. This is especially significant in the context of increasing lesion contrast and increasing retention of daughters. Even though there is increasing activity of Fr-221 or Bi-213 as lesion contrast increases or more of the daughters are retained in the lesion, the performance of DOSEM-based reconstruction degrades.

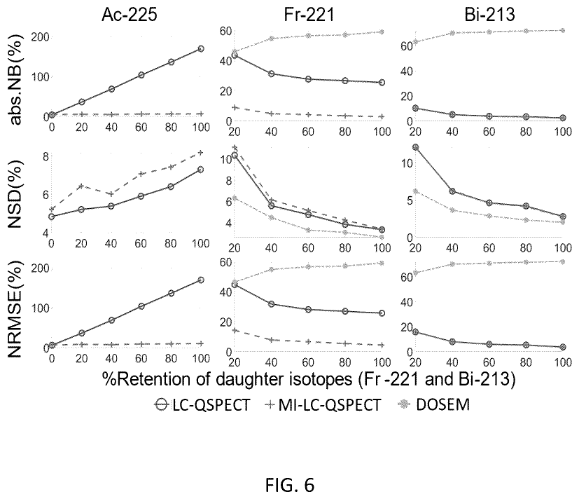

FIG. 6 illustrates the absolute NB, NSD, and NRMSE of the estimated lesion uptake of Ac-225, Fr-221, and Bi-213 as a function of % Retention of daughter isotope. It is observed the performance of LC-QSPECT degrades as more daughters are retained in the lesion while the performance of MI-LC-QSPECT stayed consistent. This demonstrates the advantage of MI-LC-QSPECT, where crosstalk contamination between the isotopes can be modeled, significantly increasing accuracy.

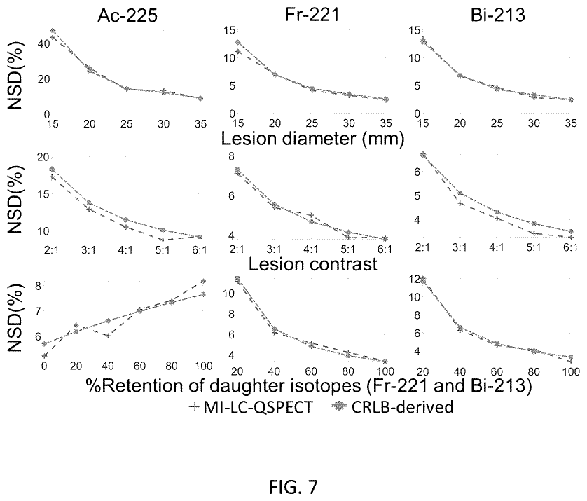

FIG. 7 illustrates the NSD of the estimates obtained with the disclosed method and that derived from the Cramer-Rao lower bound (CRLB). CRLB provides a theoretical limit on the variance/precision of an unbiased estimator. The CRLB states that for any unbiased estimator of a parameter, the variance is at least as large as the inverse of the Fisher information, I(λₖ), thus the CRLB-derived NSD of the k^{th} VOI can be defined as:

$${CRLB\_NSD}_{k} = \frac{1}{{λ}_{k}} \sqrt{\frac{1}{I ( {λ}_{k} )}} \qquad ( 20 )$$

As shown, the disclosed MI-LC-QSPECT method achieves NSDs close to the CRLB-derived NSDs, indicating how efficient the method is. The CRLB serves as a fundamental benchmark which guides the assessment of efficient estimation methods. CRLB functionally sets a limit on how precise an unbiased estimator can be and achieving the CRLB indicates that an estimator is efficient. It is important to note that the CRLB applies strictly to unbiased estimators, and it highlights the theoretical best-case scenario. Thus, it is possible to achieve lower variance when there is some bias in the estimation process.

Both PDQ methods performed better than DOSEM-based reconstruction with MI-LC-QSPECT and LC-QSPECT achieving similar performance in the case of Bi-213. It is noted that both PDQ methods estimate a much lower number of parameters, i.e. regional activity uptake in VOIs instead of estimating for large number of voxels as is done in DOSEM-based reconstruction. This significantly improves the stability and efficiency of the proposed method. Even though LC-QSPECT performed better than DOSEM-based reconstruction, it can be observed that there are significant differences in its performance for each of the isotopes. The performance of LC-QSPECT degrades as crosstalk contamination increases in the energy windows, more evidently demonstrated for the estimation of Ac-225, where LC-QSPECT achieved >100% bias.

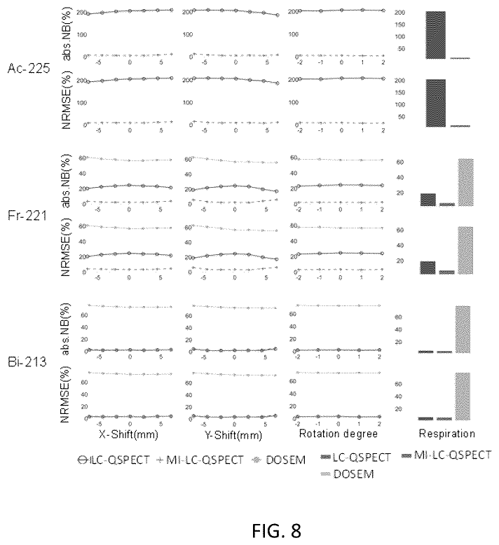

FIG. 8 illustrates the absolute NB and NRMSE of the estimated lesion uptake of Ac-225, Fr-221, and Bi-213 for all considered cases of inaccurate VOI definitions. As can be seen, MI-LC-QSPECT still outperformed the existing methods even when there are inaccurate VOI definitions caused by subject movement during imaging. Also observed is that the disclosed method has minimal sensitivity to misalignment due to linear shift in the x-axis, y-axis or angular shifts about the y-axis. Given that implementation of MI-LC-QSPECT heavily depends on accurate delineation of the VOIs, it is compelling to acknowledge that margins of errors that may typically occur in clinical settings during VOI delineation with aligned CT and SPECT images do not pose significant effect on the accuracy of the proposed method.

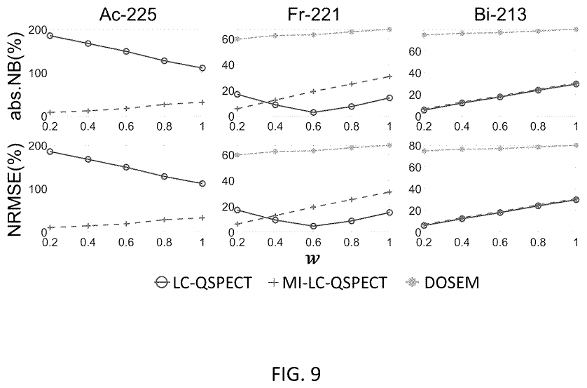

This is however a different case in the context of intra-regional heterogenous activity uptake FIG. 9 illustrates the absolute NB and NMSE of the estimated lesion uptake of Ac-225, Fr-221, and Bi-213 for various levels of heterogeneity in the activity distribution. The performance of MI-LC-QSPECT can be observed to degrade as the level of heterogeneity within the lesion increases. This result is due to fact that the disclosed method does not account for heterogeneity in the regional activity uptake distributions. It assumes a homogenous intra-regional uptake. There is also no voxelization, which means the disclosed method is unable to estimate activity uptake distributions within a VOI using MI-LC-QSPECT. Notwithstanding this, the disclosed method still exhibits higher accuracy than the reconstruction-based method which is prone to erroneous activity distribution estimates in low count settings. However, in clinical practice, reconstruction-based methods can be used in conjunction with MI-LC-QSPECT to provide physicians a graphical representation of the activity uptake distribution within a VOI, while having access to more accurate mean regional uptake estimates.

The disclosed MI-LC-QSPECT method uses a Monte Carlo-based approach to generate the system matrices, enabling accurate modeling of the SPECT physics including crosstalk contamination among the isotopes. Generating the system response matrix using the MC-based approach for the VOIs of a typical subject can be relatively fast and it can easily be stored. It also takes approximately 8 mins to perform 600 iterations of the MI-LC-QSPECT method using the MATLAB software on a computer with Intel® Core™ i7-10700 CPU and 48 GB of RAM.

Disclosed herein is a generalized multi-isotope low-count quantitative SPECT (MI-LC-QSPECT) method which directly estimates the regional activity uptake of any number of γ-emitting isotopes using the SPECT projections acquired over multiple energy windows. For a certain N number of isotopes, developed was a series of N-equations that model the crosstalk among the isotope emissions and can be solved iteratively to estimate the regional activity uptake of each isotope. This method was validated by performing activity uptake quantification for Ac-225 and its daughters Fr-221 and Bi-213 using clinically realistic simulation studies. The accuracy and precision of the method for different lesion sizes, lesion contrasts and uptake retention rates of the daughter isotopes in the lesion was then evaluated. The disclosed method demonstrated the ability to reliably estimate the mean activity uptake of Ac-225, Fr-221 and Bi-213, outperforming DOSEM based reconstruction and LC-QSPECT. Additionally, the proposed MI-LC-QSPECT method demonstrated significant improvement in quantification over the current methods in the contexts of inaccurate VOI definitions and intra-regional heterogenous uptake distribution. The results indicate that MI-LC-QSPECT provides reliable isotopic uptake estimation, advancing projection-domain quantitation methods in low-count imaging for improved targeted alpha therapy outcomes. This motivates further clinical evaluation and applications across various radionuclides.

The MI-LC-QSPECT method, with the ability to model the crosstalk among Ac-225 and its daughters, demonstrated the ability to reliably estimate the mean activity uptake of these isotopes simultaneously while outperforming existing methods. The method can also be used to ascertain if Fr-221 and Bi-213 form independent biodistributions apart from Ac-225. This method can be advanced for other radionuclides. For example, this method may be used for simultaneous quantification of the mean activity uptake of other parent isotopes including Lead-212 and Astatine-211 and their respective daughter isotopes. This study further corroborates and affirms the need for the use of projection domain quantitation methods in estimating mean activity uptake in low-count imaging.

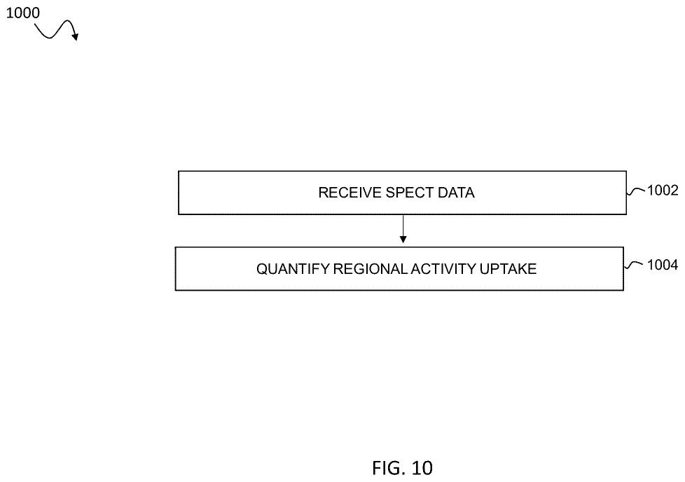

FIG. 10 illustrates a process diagram for a method 1000 for providing single-photon emission tomography (SPECT) uptake data according to the present disclosure. Method 1000 includes receiving 1002 SPECT data. Method 1000 further includes quantifying 1004 regional activity uptake. One or more of steps 1002-1004 of method 1000 may include utilizing one or more of Equations 1-20 and/or the processing pipeline as described herein.

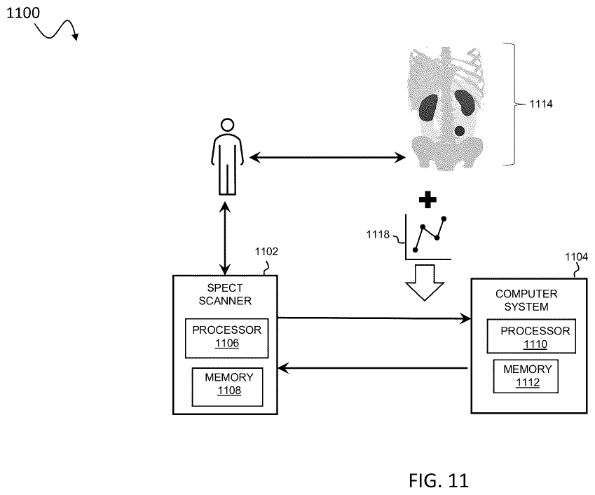

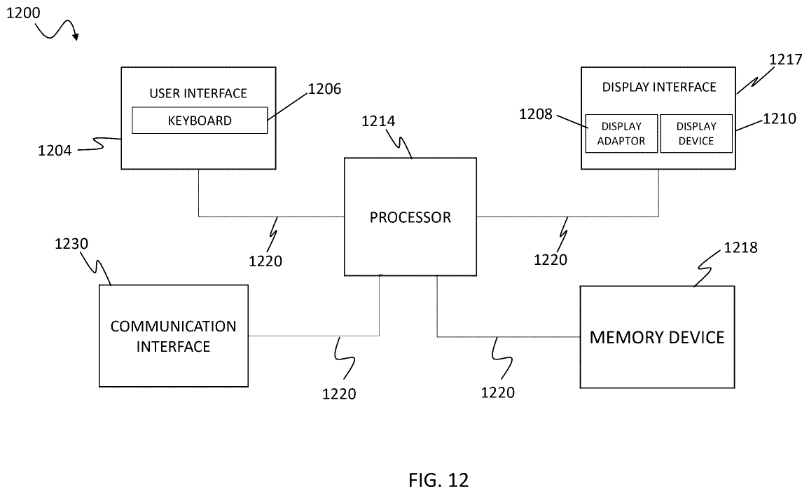

In various aspects, the methods described herein may be implemented using an SPECT system. FIG. 11 is an illustration of an SPECT system 1100 in one aspect. As illustrated in FIG. 11, the SPECT system 1100 may include an SPECT scanner 1102 operatively coupled and/or in communication with a computer system 1104. In this aspect, the computer system 1104 is configured to receive data from the SPECT scanner 1102 and is further configured to execute a plurality of stored executable instructions encoding one or more aspects of the SPECT method as described herein above. In another aspect, the computer system 1104 may be further configured to operate the SPECT scanner 1102 to obtain, for example, data by executing an additional plurality of stored executable instructions. The computer system 1104 may be located near the SPECT scanner 1102 (e.g., in the same or an adjacent room) or may be remotely located from the SPECT scanner (e.g., in a different building, a different city, a different country, etc.). Moreover, the computer system 1104 may include combinations of local and remote components and may be or include a cloud computing system.

SPECT scanner 1102 may include one or more processors 1106 and one or more memories 1108 operatively connected to the one or more processors 1106. SPECT scanner 1102 may be configured and fully equipped for advanced imaging. As described herein, SPECT scanner 1102 may be realized as a Siemens™ Symbia SPECT/CT Imaging system equipped with NaI(TI) crystals and parallel high-energy general-purpose (HEGP) collimator in some embodiments.

Computer system 1104 may include one or more processors 1110 for executing computer-readable instructions and one or more memories 1112 operatively connected to the one or more processors 1110. In this regard, each processor 1110 of system 1104 may be multi-core. Additionally, or alternatively, parallel processing may be implemented by one or more operatively connected systems 1104 in a distributed computing configuration.

In operation, SPECT scanner 1102 may be configured to obtain SPECT images 1114 of a patient/participant 1116, including all underlying information and data 1118 associated with such SPECT images 1114. Computer system 1104 may be configured to obtain or otherwise receive SPECT images 1114 and corresponding information and data 1118 (e.g., uptake data) obtained from one or more SPECT scanners 1102. SPECT images 1114 and information and data 1118, as well as corresponding calculated data as described herein, may be stored in a memory 1112.

The systems and methods described herein may be implemented in any suitable computing device 1200 and software implemented therein. FIG. 12 is a block diagram of an example computing device 1200. In the example embodiment, the computing device 1200 includes a user interface 1204 that receives at least one input from a user. The user interface 1204 may include a keyboard 1206 that enables the user to input pertinent information. The user interface 1204 may also include, for example, a pointing device, a mouse, a stylus, a touch sensitive panel (e.g., a touch pad and a touch screen), a gyroscope, an accelerometer, a position detector, and/or an audio input interface (e.g., including a microphone).

Moreover, in the example embodiment, computing device 800 includes a display interface 1217 that presents information, such as input events and/or validation results, to the user. The display interface 1217 may also include a display adapter 808 that is coupled to at least one display device 1210. More specifically, in the example embodiment, the display device 1210 may be a visual display device, such as a cathode ray tube (CRT), a liquid crystal display (LCD), a light-emitting diode (LED) display, and/or an “electronic ink” display. Alternatively, the display interface 1217 may include an audio output device (e.g., an audio adapter and/or a speaker) and/or a printer.

The computing device 1200 also includes a processor 1214 and a memory device 1218. The processor 1214 is coupled to the user interface 804, the display interface 1217, and the memory device 1218 via a system bus 1220. In the example embodiment, the processor 1214 communicates with the user, such as by prompting the user via the display interface 1217 and/or by receiving user inputs via the user interface 1204. The term “processor” refers generally to any programmable system including systems and microcontrollers, reduced instruction set computers (RISC), complex instruction set computers (CISC), application specific integrated circuits (ASIC), programmable logic circuits (PLC), and any other circuit or processor capable of executing the functions described herein. The above examples are example only, and thus are not intended to limit in any way the definition and/or meaning of the term “processor.”

In the example embodiment, the memory device 1218 includes one or more devices that enable information, such as executable instructions and/or other data, to be stored and retrieved. Moreover, the memory device 1218 includes one or more computer readable media, such as, without limitation, dynamic random access memory (DRAM), static random access memory (SRAM), a solid state disk, and/or a hard disk. In the example embodiment, the memory device 1218 stores, without limitation, application source code, application object code, configuration data, additional input events, application states, assertion statements, validation results, and/or any other type of data. The computing device 1200, in the example embodiment, may also include a communication interface 1230 that is coupled to the processor 1214 via the system bus 1220. Moreover, the communication interface 1230 is communicatively coupled to data acquisition devices.

In the example embodiment, the processor 1214 may be programmed by encoding an operation using one or more executable instructions and providing the executable instructions in the memory device 1218. In the example embodiment, the processor 814 is programmed to select a plurality of measurements that are received from data acquisition devices.

In operation, a computer executes computer-executable instructions embodied in one or more computer-executable components stored on one or more computer-readable media to implement aspects of the invention described and/or illustrated herein. The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

Example embodiments of systems and methods of adaptive radiotherapy are described above in detail. The systems and methods are not limited to the specific embodiments described herein but, rather, components of the systems and/or operations of the methods may be utilized independently and separately from other components and/or operations described herein. Further, the described components and/or operations may also be defined in, or used in combination with, other systems, methods, and/or devices, and are not limited to practice with only the systems described herein.

Although the present disclosure is described in connection with an exemplary imaging system environment, embodiments of the invention are operational with numerous other general purpose or special purpose imaging system environments or configurations. The imaging system environment is not intended to suggest any limitation as to the scope of use or functionality of any aspect of the invention. Moreover, the imaging system environment should not be interpreted as having any dependency or requirement relating to any one or combination of components illustrated in the exemplary operating environment. Examples of well-known imaging systems, environments, and/or configurations that may be suitable for use with aspects of the invention include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

As used herein, the term “database” may refer to either a body of data, a relational database management system (RDBMS), or to both. As used herein, a database may include any collection of data including hierarchical databases, relational databases, flat file databases, object-relational databases, object oriented databases, and any other structured collection of records or data that is stored in a computer system. The above examples are example only, and thus are not intended to limit in any way the definition and/or meaning of the term database. Examples of RDBMS's include, but are not limited to including, Oracle® Database, MySQL, IBM@DB2, Microsoft® SQL Server, Sybase®, and PostgreSQL. However, any database may be used that enables the systems and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California.)

In one embodiment, a computer program is provided to enable the data processing of the SPECT method as described herein above, and this program is embodied on a computer readable medium. In an example embodiment, the computer system is executed on a single computer system, without requiring a connection to a server computer. In a further embodiment, the computer system is run in a Windows® environment (Windows is a registered trademark of Microsoft Corporation, Redmond, Washington). In yet another embodiment, the computer system is run on a mainframe environment and a UNIX® server environment (UNIX is a registered trademark of X/Open Company Limited located in Reading, Berkshire, United Kingdom). Alternatively, the computer system is run in any suitable operating system environment. The computer program is flexible and designed to run in various different environments without compromising any major functionality. In some embodiments, the computer system includes multiple components distributed among a plurality of computing devices. One or more components may be in the form of computer-executable instructions embodied in a computer-readable medium.

The computer systems and processes are not limited to the specific embodiments described herein. In addition, components of each computer system and each process can be practiced independent and separate from other components and processes described herein. Each component and process also can be used in combination with other assembly packages and processes.

It will be understood by those of skill in the art that information and signals may be represented using any of a variety of different technologies and techniques. For example, data, instructions, commands, information, signals, bits, symbols, and/or chips may be represented by voltages, currents, electromagnetic waves, magnetic fields or particles, optical fields or particles, or any combination thereof. Similarly, the various illustrative logical blocks, modules, circuits, and algorithm operations described herein may be implemented as electronic hardware, computer software, or a combination of both, depending on the application and the functionality. Moreover, the various logical blocks, modules, and circuits described herein may be implemented or performed with a general purpose computer, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. Exemplary general purpose processors include, but are not limited to only including, microprocessors, conventional processors, controllers, microcontrollers, state machines, or a combination of computing devices.

When introducing elements of aspects of the invention or embodiments thereof, the articles “a,” “an,” “the,” and “said” are intended to mean that there are one or more of the elements. The terms “comprising,” including,” and “having” are intended to be inclusive and mean that there may be additional elements other than the listed elements.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

What is claimed is:

1. A computer system for providing single-photon emission tomography (SPECT) uptake data, the computer system including at least one processor in communication with at least one memory device, the at least one processor programmed to:
receive SPECT data obtained from one or more SPECT scanners configured to perform at least one SPECT acquisition, the SPECT data including uptake information associated with the at least one SPECT acquisition; and
quantify regional activity uptake of at least one isotope of a plurality of isotopes based on the uptake activity.

2. The computer system of claim 1, wherein the at least one isotope comprises a parent gamma-particle emitting isotope and/or at least one daughter gamma-particle emitting isotope.

3. The computer system of claim 2, wherein the at least one parent gamma-particle emitting isotopes is selected from the group comprising of Actinium-225, Lead-212, Astatine-211, or other gamma-photon emitting agents in alpha-particle radiopharmaceutical therapies.

4. The computer system of claim 1, wherein the regional activity uptake is mean activity uptake.

5. The computer system of claim 1, wherein the SPECT data is low-count SPECT data.

6. The computer system of claim 1, wherein the at least one processor is further programmed to:
generate a graphical representation of the activity uptake within a volume-of-interest; and
display the graphical representation of the activity uptake within a volume-of-interest.

7. The computer system of claim 1, wherein the at least one processor is further programmed to:
quantify regional activity uptake of at least two isotopes of a plurality of isotopes simultaneously based on the uptake activity.

8. A computer-implemented method for providing single-photon emission tomography (SPECT) uptake data, using at least one processor in communication with at least one memory device, the method comprising:
receiving SPECT data obtained from one or more SPECT scanners configured to perform at least one SPECT acquisition, the SPECT data including uptake information associated with the at least one SPECT acquisition; and
quantifying regional activity uptake of at least one isotope of a plurality of isotopes based on the uptake activity.

9. The computer-implemented method of claim 8, wherein the at least one isotope comprises a parent gamma-particle emitting isotope and/or at least one daughter gamma-particle emitting isotope.

10. The computer-implemented method of claim 9, wherein the at least one parent gamma-particle emitting isotopes is selected from the group comprising of Actinium-225, Lead-212, Astatine-211, or other gamma-photon emitting agents in alpha-particle radiopharmaceutical therapies.

11. The computer-implemented method of claim 8, wherein the regional activity uptake is mean activity uptake.

12. The computer-implemented method of claim 8, wherein the SPECT data is low-count SPECT data.

13. The computer-implemented method of claim 8, wherein the method further comprises:
generating a graphical representation of the activity uptake within a volume-of-interest; and
displaying the graphical representation of the activity uptake within a volume-of-interest.

14. One or more non-transitory computer-readable storage media for a computing system providing single-photon emission tomography (SPECT) uptake data, the one or more non-transitory computer-readable storage media comprising a plurality of instructions stored thereon that, in response to being executed, cause the computing system to:
receive SPECT data obtained from one or more SPECT scanners configured to perform at least one SPECT acquisition, the SPECT data including uptake information associated with the at least one SPECT acquisition; and
quantify regional activity uptake of at least one isotope of a plurality of isotopes based on the uptake activity.

15. The one or more non-transitory computer-readable storage media of claim 14, wherein the at least one isotope comprises a parent gamma-particle emitting isotope and/or at least one daughter gamma-particle emitting isotope.

16. The one or more non-transitory computer-readable storage media of claim 15, wherein the at least one parent gamma-particle emitting isotopes is selected from the group comprising of Actinium-225, Lead-212, Astatine-211, or other gamma-photon emitting agents in alpha-particle radiopharmaceutical therapies.

17. The one or more non-transitory computer-readable storage media of claim 14, wherein the regional activity uptake is mean activity uptake.

18. The one or more non-transitory computer-readable storage media of claim 14, wherein the SPECT data is low-count SPECT data.

19. The one or more non-transitory computer-readable storage media of claim 14, wherein the plurality of instructions, in response to being executed, further causes the computing system to:
generate a graphical representation of the activity uptake within a volume-of-interest; and
display the graphical representation of the activity uptake within a volume-of-interest.

20. The one or more non-transitory computer-readable storage media of claim 14, wherein the plurality of instructions, in response to being executed, further causes the computing system to:
quantify regional activity uptake of at least two isotopes of a plurality of isotopes simultaneously based on the uptake activity.
